(19) United States
(12) Patent Application Publication
(10) Pub. No.: US 2024/0122184 A1
(43) Pub. Date: Apr. 18, 2024

(54) SYSTEMS AND METHODS FOR IDENTIFYING GAS CONCENTRATIONS USING AN MOX SENSOR

(71) Applicant: Chorus, LLC, Marlborough, MA (US)

(72) Inventors: Richard S. Rader, Wayland, MA (US); Colin J. Holmes, Delafield, WI (US)

(73) Assignee: Chorus, LLC, Marlborough, MA (US)

(21) Appl. No.: 18/395,587

(22) Filed: Dec. 24, 2023

Related U.S. Application Data
(63) Continuation-in-part of application No. 18/189,451, filed on Mar. 24, 2023.
Application No. 18/189,451 is a continuation of application No. 18/146,628, filed on Dec. 27, 2022.
Application No. 18/146,628 is a continuation of application No. 17/567,117, filed on Jan. 1, 2022, now Pat. No. 11,533,914.
Application No. 17/567,117 is a continuation-in-part of application No. PCT/US2021/036501, filed on Jun. 8, 2021.
(60) Provisional application No. 63/036,412, filed on Jun. 8, 2020.
(60) Provisional application No. 63/049,524, filed on Jul. 8, 2020.
(60) Provisional application No. 63/049,541, filed on Jul. 8, 2020.
(60) Provisional application No. 63/049,919, filed on Jul. 9, 2020.
(60) Provisional application No. 63/081,459, filed on Sep. 22, 2020.
(60) Provisional application No. 63/126,734, filed on Dec. 17, 2020.
(60) Provisional application No. 63/157,368, filed on Mar. 5, 2021.
(60) Provisional application No. 63/477,113, filed on Dec. 23, 2022.
(60) Provisional application No. 63/477,117, filed on Dec. 23, 2022.
(60) Provisional application No. 63/599,032, filed on Nov. 15, 2023.

(51) Int. Cl. A01N 59/00; A61L 2/20; A61L 2/24; A61L 9/015; A61L 9/14; A61P 31/04; A61P 31/12; G16H 50/20; G16H 50/80; G16H 70/60

(52) U.S. Cl. CPC A01N 59/00; A61L 2/20; A61L 2/24; A61L 9/015; A61L 9/14; A61P 31/04; A61P 31/12; G16H 50/20; G16H 50/80; G16H 70/60; A61L 2101/06; A61L 2209/16

(57) ABSTRACT

Metal oxide sensors are provided. Systems including the metal oxide sensors and methods for identifying a target gas concentration using the sensors are also provided. A method for increasing the consistency of MOx sensor readings over an in-service interval is provided, comprising: applying a thermal stimulus to a sensor face to reset to a condition substantially devoid of environmental chemical species; a dwell period following the thermal stimulus where the sensor is: (a) held between an ambient temperature and a thermal stimulus temperature, or (b) exposed to temporal patterns of heated and unheated intervals; triggering a reading of electrochemical changes to the sensor caused by chemical species in the environment being sensed, at a consistent time interval after the application of the thermal stimulus; and wherein the application of the thermal stimulus, the dwell period, and the reading of electrochemical changes to the sensor is a thermal stimulus-to-read cycle.

### CROSS-REFERENCE TO RELATED APPLICATIONS

This application is a continuation-in-part of U.S. patent application Ser. No. 18/189,451, filed on Mar. 24, 2023, which is a continuation of U.S. patent application Ser. No. 18/146,628, filed on Dec. 27, 2022, which is a continuation of U.S. patent application Ser. No. 17/567,117, filed on Jan. 1, 2022, issued as U.S. Pat. No. 11,533,914, which is a continuation-in-part of PCT Patent Application No. PCT/US2021/036501, filed on Jun. 8, 2021, which claims priority from U.S. Provisional Patent Application No. 63/036,412, filed on Jun. 8, 2020, U.S. Provisional Patent Application No. 63/049,524, filed on Jul. 8, 2020, U.S. Provisional Patent Application No. 63/049,541, filed on Jul. 8, 2020, U.S. Provisional Patent Application No. 63/049,919, filed on Jul. 9, 2020, U.S. Provisional Patent Application No. 63/081,459, filed on Sep. 22, 2020, U.S. Provisional Patent Application No. 63/126,734, filed on Dec. 17, 2020, and U.S. Provisional Patent Application No. 63/157,368, filed on Mar. 5, 2021. This application also claims priority from U.S. Provisional Patent Application No. 63/477,113, filed on Dec. 23, 2022, U.S. Provisional Patent Application No. 63/477,117, filed on Dec. 23, 2022, and U.S. Provisional Patent Application No. 63/599,032, filed on Nov. 15, 2023. Each of these applications is incorporated by reference herein in its entirety.

### BACKGROUND

Infectious diseases such as human immunodeficiency virus and acquired immune deficiency syndrome (HIV/AIDS), tuberculosis (TB), severe acute respiratory syndrome (SARS-CoV-1), Ebola virus disease (EVD), and coronavirus disease 2019 (COVID-19) are contagious diseases transmissible through direct contact from person to person, through indirect contact by breathing airborne droplets spread from an infected person, and through contact with surfaces of contaminated objects.

With the COVID-19 pandemic outbreak, facemask or respirator wearing and practicing social distancing may mitigate airborne droplets spread by potential neighboring human carriers. Nevertheless, both of these practices are defensive actions that do not destroy or disinfect the germs or viruses in the airborne droplets. Currently, methods that are used to generate antimicrobial gases or vapor are large and impractical for general household or office use or for personal use in a limited localized space, and methods of generating ClO₂ from liquid and solid precursor chemicals are slow and/or generate low quality ClO₂ solutions.

Antimicrobial gas, such as chlorine dioxide (ClO₂), has demonstrated capability as an antimicrobial or inactivator for pathogens on hard surfaces. In gas form, ClO₂ has demonstrated capability to disinfect hard surfaces and porous materials within three-dimensional spaces. ClO₂ gas has also been shown to kill or otherwise inactivate airborne pathogens, and even protect against airborne contagion.

However, accurately and quickly measuring the concentration of a target gas, including an antimicrobial gas, to ensure that the target gas is not above acceptable values requires precise sensors capable of identifying the target gas in a few parts per billion of atmospheric gas. Sensors, such as metal oxide (MOx) sensors, which include both analog metal oxides on substrates and MOx layers integrated with primarily analog to digital convert (semiconductors metal oxide or “SMOx”) sensors, may be particularly useful for such an application due to the small size and relatively low cost of these sensors. An issue with these sensors is a lack of accuracy over time due to sensor drift.

A unique aspect of MOx sensors is that MOx sensors only interact with specific gaseous species at elevated temperatures (typically above 100 degrees Celsius) requiring a heating element adjacent to the MOx layer to generate temperatures typically up to or near 500 degrees Celsius. The methods to improve MOx sensor stability and sensor performance via programmatic and repeatable control of temporal patterns of thermal excursions between ambient to chemically interactive MOx layer temperatures via the adjacent heating element are described herein.

The present disclosure relates to a method to increase the stability of MOx sensor electronic readings over time spans greater than seconds, methods for controlling the MOx sensors to achieve said stability, and using both to enable accurate qualitative or quantitative measurement of a target gas concentration by mathematical transform of the electronic sensor reading to concentration of a gas.

### SUMMARY

In one aspect, a method for increasing the consistency of MOx sensor readings over an in-service interval spanning minutes to years is provided, the method comprising: applying a thermal stimulus to a MOx sensor face to reset the sensor face to a consistent condition that substantially removes accumulated environmental chemical species on the sensor face in the past; a dwell period following the application of the thermal stimulus where the MOx sensor is at least one of: (a) held at a temperature between an ambient temperature and a thermal stimulus temperature, or (b) exposed to temporal patterns of heated and unheated intervals; subsequent to the dwell period triggering a reading of electrochemical changes to the MOx sensor caused by chemical species in the environment being sensed by the MOx sensor, at a consistent time interval after the application of the thermal stimulus; and wherein the application of the thermal stimulus, the following dwell period, and the reading of electrochemical changes to the MOx sensor is a single thermal stimulus-to-read cycle.

In one aspect, a system for identifying a target gas concentration using an MOx sensor is provided, the system comprising: a metal oxide sensor having: a substrate supporting a sintered powder MOx surface coating, one or more electrode electrically connected to the surface coating, a heater that is integrated or attached to the substrate supporting the MOx surface coating, and a power source; a heater controller; and a system controller; wherein the system controller is operatively connected to the heater controller and configured to cause the heater to input heat to the surface coating in a continuous cycle of clean, dwell, and read actions, wherein the clean action includes heating the surface coating to a temperature sufficient to remove adsorbed or absorbed water, target gases, and other environmental gases on the surface coating but below a level that would change a sintered material characteristic of the surface coating, wherein the dwell action includes periodically heating the surface coating to a temperature less than the clean action temperature, and wherein the read action includes heating the surface temperature to a temperature that is also less than the clean action temperature.

In another aspect, a method for identifying a target gas concentration using an MOx sensor is provided, the method comprising: providing a metal oxide sensor having: a substrate supporting a sintered powder MOx surface coating, one or more electrode electrically connected to the surface coating, a heater, and a power source; providing a heater controller; and providing a system controller; wherein the system controller is operatively connected to the heater controller and causes the heater to input heat to the surface coating in a continuous cycle of clean, dwell, and read actions, wherein the clean action includes heating the surface coating to remove adsorbed or absorbed water, target gasses, and other environmental gasses on the surface coating but below a level that would change a sintered material characteristic of the surface coating, wherein the dwell action includes periodically heating the surface coating to a temperature less than the clean action temperature, and wherein the read action includes heating the surface temperature to a temperature that is also less than the clean action temperature.

### BRIEF DESCRIPTION OF THE FIGURES

FIG. 1A illustrates a sectional schematic of an example MOx sensor 102 for monitoring a target gas.

FIG. 1B illustrates a sectional schematic of MOx sensor 102 for monitoring a target gas.

FIG. 1C illustrates a sectional schematic of MOx sensor 102 for monitoring a target gas.

FIG. 1D illustrates an example arrangement of MOx sensor 102 for monitoring a target gas.

FIG. 2 illustrates a diagram of an example system 200 for identifying target gas concentrations using an MOx sensor 202.

FIG. 3A illustrates a schematic of an example system 300 for identifying target gas concentrations using an MOx sensor 302.

FIG. 3B illustrates a schematic of system 300 for identifying target gas concentrations using MOx sensor 302.

FIG. 4 illustrates an example blueprint of a network 432 of target gas sensor systems 400 and sensors distributed in rooms and spaces within a floor of a building.

FIG. 5 illustrates a graph plotting a response signal of a traditional MOx sensor as a function of operating time.

FIG. 6 illustrates a graph showing MOx sensor drift from sensor surface changes.

FIG. 7 illustrates a schematic of MOx sensor heater settings for clean-dwell-read cycling of the sensor face.

FIG. 8A illustrates a variation of clean-dwell-read cycling of the sensor face.

FIG. 8B illustrates a variation of clean-dwell-read cycling of the sensor face.

FIG. 8C illustrates a variation of clean-dwell-read cycling of the sensor face.

FIG. 8D illustrates a variation of clean-dwell-read cycling of the sensor face.

FIG. 8E illustrates a variation of clean-dwell-read cycling of the sensor face.

FIG. 9A illustrates a potential sensor response to a variation of clean-dwell-read cycling of the sensor face.

FIG. 9B illustrates a potential sensor response to a variation of clean-dwell-read cycling of the sensor face.

FIG. 9C illustrates a potential sensor response to a variation of clean-dwell-read cycling of the sensor face.

FIG. 9D illustrates a potential sensor response to a variation of clean-dwell-read cycling of the sensor face.

FIG. 10 illustrates a graph plotting the time-based recovery of the surface of an MOx sensor to long term baseline after a condition cycle has been completed until 275 seconds has passed.

FIG. 11 illustrates a graph showing MOx sensor stability over seven days of a stability experiment.

FIG. 12 illustrates a graph showing MOx sensor stability as a result of conditioning cycles.

FIG. 13 illustrates an array of MOx sensors 1302, each using a different variation of clean-dwell-read cycling of the sensor face.

### DETAILED DESCRIPTION

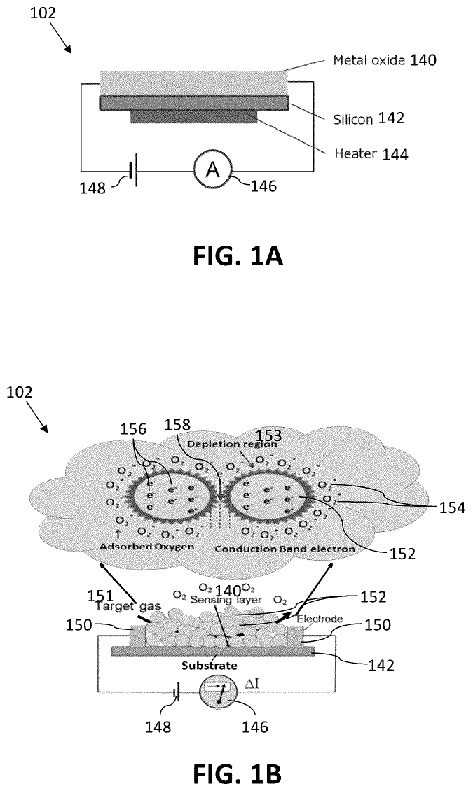

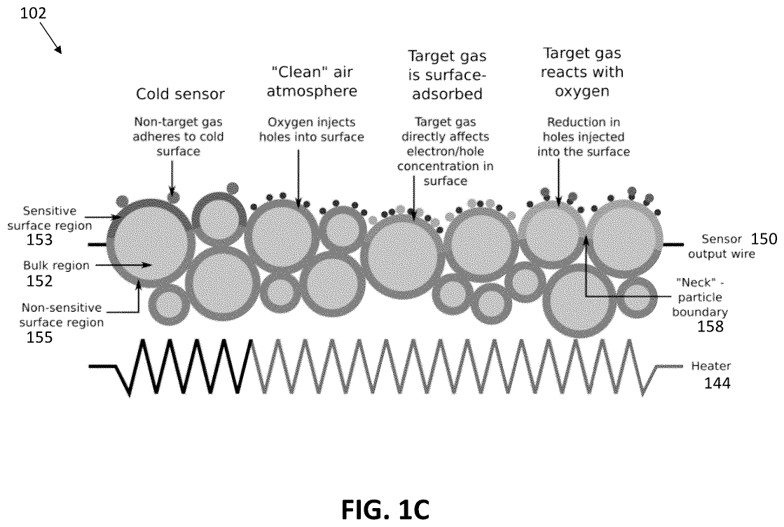

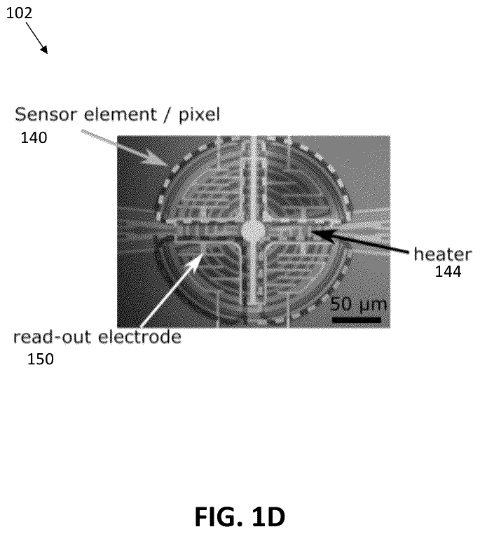

FIGS. 1A-1D illustrate an example MOx sensor 102 for monitoring a target gas. MOx sensor 102, including the subset of digital semiconductor SMOx sensors, are based upon the interaction of gaseous species with the heated surface coating 140 of a composite structure within the sensor housing or packaging.

Surface coating 140 may be formed from an MOx material. Surface coating 140 may be formed from a sintered powder MOx material. Surface coating 140 may be in contact with the ambient air, and thus the target gas to be sensed by sensor 102. Surface coating 140 may overlay a substrate 142. Substrate 142 may be formed from a silicon, ceramic, or other supportive material. The combination of surface coating 140 and substrate 142 may form the composite structure.

MOx sensor 102 may include a heater 144. Heater 144 may be oriented in contact with substrate 142 and/or surface coating 140. Heater 144 may be oriented in contact with substrate 142, and heat may conduct through substrate 142 to heat surface coating 140. Heater 144 may be capable of heating surface coating 140 up to hundreds of degrees Celsius. For example, heater 144 may be capable of heating surface coating 140 to at least 300 degrees Celsius, 400 degrees Celsius, 500 degrees Celsius, and the like. Heater 144 may use convection or radiant heating methods to activate gas interaction at surface coating 140.

Sensor 102 may include circuitry, which may include an ammeter 146 to measure current in the circuitry. Ammeter 146 may alternatively be an electronic system to measure voltage, resistance, capacitance, or other electrical characteristics of surface coating 140. The circuitry may include a power source 148. Sensor 102 may include one or more electrode 150 electrically connected to the circuitry, and one or both of surface coating 140 and substrate 142. The circuitry may be configured to provide electrical sensing across sensing layer 140 (which may include a metal oxide material).

Sensor 102 may include a stable oxide layer/bulk region 152 formed on surface coating 140. Stable oxide layer 152 may interact with a reactive target gas 151 contained in the ambient air. Stable oxide layer 152 may adsorb oxygen 154 in a depletion region/sensitive surface region 153. Stable oxide layer 152 may include conduction band electrons 156. Stable oxide layer 152 may include a particle boundary 158 between particles of stable oxide layer 152. Stable oxide layer 152 may include a non-sensitive surface region 155.

FIG. 1C illustrates sensor 102 in four different conditions as sensor 102 is heated by heater 144. Starting at the leftmost position (first position), sensor 102 is cold (e.g., not heated), and non-target gases adhere to the cold surface of stable oxide layer 152. Sensor 102 exhibits a base resistance in this condition. This condition represents a “null concentration” of reactive species of gas.

In the second position, sensor 102 is heated by heater 144, and oxygen injects holes into the surface of stable oxide layer 152. This condition represents a “null concentration” of reactive species of gas.

In the third position, sensor 102 remains heated by heater 144, and a target gas (e.g., an antimicrobial gas, such as ClO₂) is adsorbed into the surface of stable oxide layer 152, which directly affects the electron/hole concentration in the surface of stable oxide layer 152. This condition represents the interaction of active target species with the oxygenated hot layer and particle boundary.

Finally, in the rightmost position (fourth position), sensor 102 remains heated by heater 144, and the target gas reacts with oxygen resulting in a reduction of holes injected into the surface of stable oxide layer 152. This condition represents the interaction of active target species with the oxygenated hot layer and particle boundary.

Sensor 102's output is the resistance across the whole of the sensor material, which forms a resistor network with contributions from both the bulk and surface regions of stable oxide layer 152. As a result, variation in base resistance between individual sensors 102 is the result of the random nature of the surface geometry of individual sensors 102.

MOx sensors 102 are utilized for trace to low-concentration detection of target gases. Thus, the concentrations of analytes are orders of magnitude lower than the atmosphere's 21% concentration of oxygen. Sensors 102 are particularly sensitive because at temperatures of hundreds degrees Celsius, oxygen reacts with both/either the MOx surface coating 140 and/or the particle boundaries 158 between the sintered particles of the stable oxide layer 152.

Target gas 151 must be reactive to be sensed by MOx sensors 102. For example, MOx sensors 102 doped for oxidizing species are insensitive to CO₂. Target gas 151 may be an oxidizing or reducing chemical/agent. Target gas 151 may be an antimicrobial gas. Target gas 151 may be ClO₂ gas. Reactive species, whether oxidized or reduced, interact with the oxygenated hot MOx surface coating 140, as shown in the fourth position of FIG. 1C.

FIG. 1D illustrates sensor 102 having a surface coating 140 divided into four separate sensor elements, a heater 144, and at least one readout electrode 150. Each sensor element may include its own readout electrode 150. Heater 144 may be arranged in a spiral or concentric circles about each sensor element, or about the entirety of sensor 102. Sensor 102 may have a diameter of between 1 and 50,000 microns.

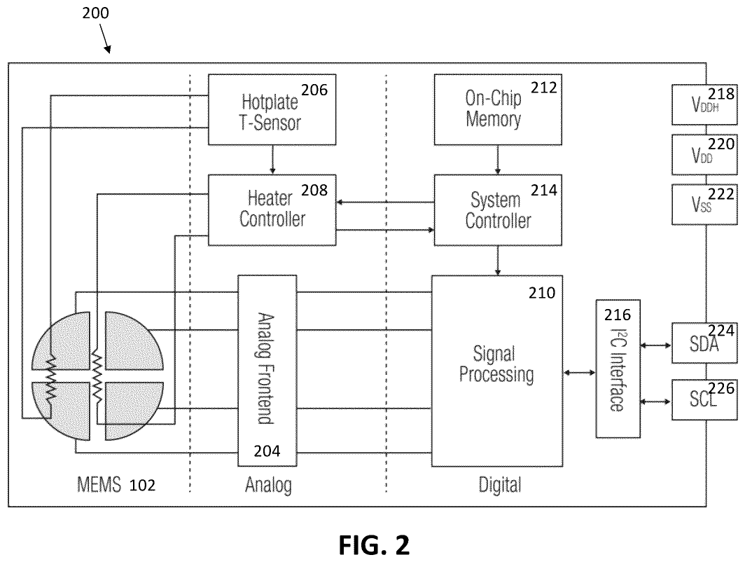

FIG. 2 illustrates a schematic of an example system 200 for identifying target gas concentrations using an MOx sensor 202. MOx sensor 202, with other components of system 200, may form a complementary metal oxide semiconductor (CMOS) MOx sensor. System 200 includes an MOx sensor 102 operatively connected to an analog frontend 204, a hotplate temperature sensor 206, and a heater controller 208. Hotplate temperature sensor 206 and a heater controller 208 are operatively connected to one another and configured to adjust the temperature of heater 144 described above. These elements make up an analog MOx sensor 102. Integration of the components of example system 200 enable coordination of timing, duration, and setpoints for heating of the active layer of the MOx sensor 202, and further synchronize when analog electronic reading(s) are taken relative to a heated or un-heated condition.

Additional elements may be added to make up a digital MOx sensor 102. A signal processing unit 210 may be operatively connected to analog frontend 204. An on-chip memory 212 may be operatively connected to a system controller 214, which is operatively connected to heater controller 208; system controller 214 may be operatively connected to signal processing unit 210. System 200 may include an I2C (inter-integrated circuit) interface 216 operatively connected to signal processing unit 210. An SDA (serial data) 224 may be operatively connected to I2C Interface 216. A SCL (serial clock) 226 may be operatively connected to I2C Interface 216. System 200 may additionally include V DDH 218 (hotplate supply voltage), V_{DD} 220 (supply voltage), and V_{SS} 222 (ground voltage). Signal processing unit 210 may interpret signals or commands exchanged via interface 216 to alter the timing, duration, and heater temperature setpoints in a programmatic sequence and synchronize the quantity and timing of readings to reflect changes in sensor surface coating 140 evoked by the diversity of time-and-temperature patterns of heating that can be imagined.

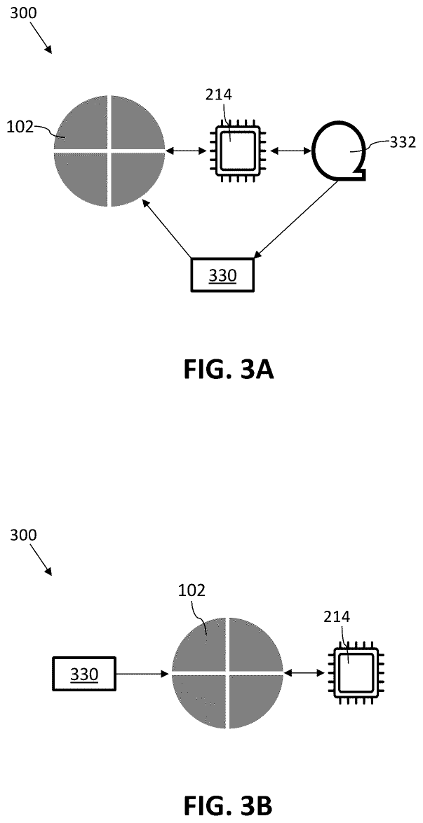

FIGS. 3A-3B illustrates a schematic of an example system 300 for identifying target gas concentrations using MOx sensor 102.

As illustrated in FIG. 3A, system 300 includes MOx sensor 102 operatively connected to a system controller 214, which is operatively connected to an antimicrobial generator 332. System 300 includes a VUT 330 (volume under treatment) into which antimicrobial generator 332 dispenses a gaseous antimicrobial. MOx sensor 102 is operatively connected to VUT 330 and can determine the concentration of antimicrobial gas within VUT 330.

As illustrated in FIG. 3B, system 300 may include MOx sensor 102, system controller 214, and VUT 330. Sensor 102 may be operatively connected to VUT 330 and system controller 214. MOx sensor 102 is operatively connected to VUT 330 and can determine the concentration of a target gas within VUT 330.

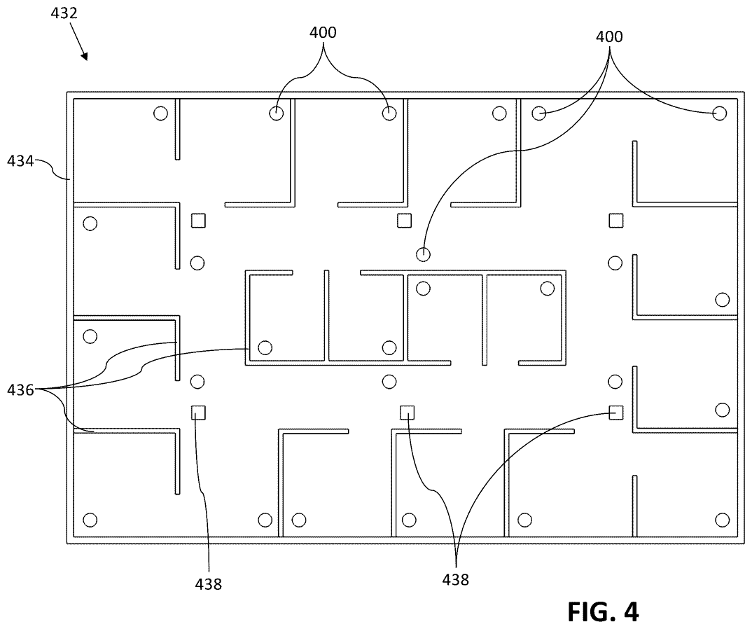

FIG. 4 illustrates an example blueprint of a network 432 of target gas sensor systems 400 and sensors distributed in rooms and spaces within a floor of a building. Network 432 illustrates a floor of a building bounded by exterior walls 434 and divided by interior walls 436. Gas generator systems 438 may include antimicrobial gas (e.g., ClO₂ gas) concentration sensors, including for example MOx sensors. As illustrated, gas generator systems 438 may be oriented in each individual room of the floor, as well as in open spaces between the individual rooms. Standalone target gas sensors 400 (configured simply to sense the concentration of an antimicrobial gas, such as ClO₂ gas, in the ambient air) supplement network 432 to ensure that the target concentration is achieved throughout network 432.

The various gas generator systems 438 may operate to generate antimicrobial gas (e.g., ClO₂ gas) independent of one another, and at different concentration target values depending upon the desired function of a particular gas generator systems 438.

For example, where a room is occupied by a patient (e.g., in a hospital or nursing facility), employee (e.g., in an office), a guest (e.g., in a hotel), or the like, the gas generator system 438 in that particular room may have a target antimicrobial gas (e.g., ClO₂ gas) concentration of about 50 ppb. After the room is no longer occupied (e.g., patient is moved from the room for a set period of time, employee is gone for the night, guest checks out, etc.), the gas generator system 438 in that room may increase its target antimicrobial gas (e.g., ClO₂ gas) concentration to about 1,000 ppb to about 5,000 ppb for a set period of time. In this manner, the room can be decontaminated (1,000 ppb to 5,000 ppb concentration level, or 50,000 ppb to 300,000 ppb concentration level for extreme pathogens) between its use by particular individuals, or on a regular time schedule, and maintain a lower safe (to humans) concentration of 50 ppb for prevention or mitigation of virus spreading while occupied. Target gas sensor systems 400 are designed to be sensitive to target gases at these concentrations.

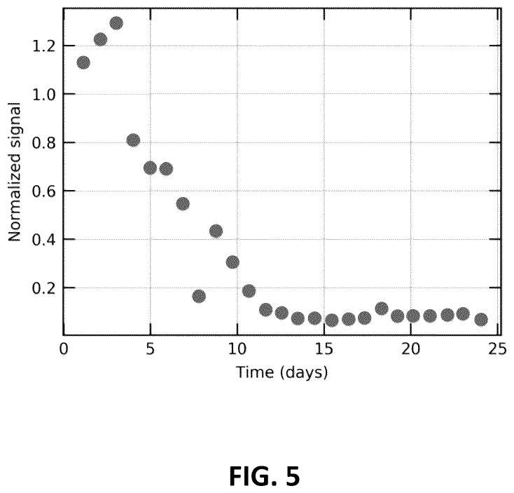

FIG. 5 illustrates a graph plotting a response signal of a traditional MOx sensor (e.g., MOx sensor 102) as a function of operating time. The surface state of an MOx sensor is dependent upon the atmosphere around the MOx sensor, regardless of the temperature of the surface coating/sensor face. Materials may adsorb or absorb onto the sensor face and some of these contaminants may remain even after temperature cycling for sensing.

Additionally, or alternatively, MOx sensors constantly exposed to any substantive oxidizing or reducing agent for time spans on the order of days lose sensitivity to the analytes that the MOx sensor is targeting, causing a quenching of sensor interaction changes over time. While this quenching may be manageable on the order of a day or two, by the time a week has passed the MOx sensor may have so much quenching that the MOx sensor is reporting a concentration of (for example) 50% of the actual concentration. In a closed loop control system, where the MOx sensor is targeting maintaining a target concentration of an antimicrobial gas may double the concentration of the controlled target gas in the room to 200% of the desired concentration.

The graph illustrated in FIG. 5 shows a traditional MOx sensor experiencing erroneous concentration readings due to quenching of the reaction of the MOx and the target analyte, which in this case is ethanol. The graph plots a normalized signal of a traditional MOx sensor to 0.5 ppm ethanol as a function of operating time, which is about 24 days. After 24 days, the sensitivity to ethanol is close to zero, while in reality, the MOx sensor is still exposed to ethanol at a concentration of 0.5 ppm.

Sensor drift has many causes, and can present as short term drift (for purposes of this discussion within one day) and long term drift (greater than a day). One objective of the system described herein is to significantly reduce short term drift and long term drift by synchronizing MOx heated sensor-gas interactions to a cycle that (a) heats the MOx layer to temperature known to remove most adsorbed chemical species without damaging the sensor; then (b) wait a period of time (dwell period); during which the system (c) assumes that the kinetics of gasses present in the environment repopulating the now “cleaned” sensor surface during the dwell period is consistent over a long enough dwell period of seconds to minutes so that; at a specific time (d) at the end of dwell the reading is taken at a consistent time interval after the dwell. Consistently presenting a “clean” (objective is to present a consistent sensor surface vs defining how “clean” the sensor surface is) starting MOx sensor surface, combined with reading the temporal (kinetic) changes in the MOx sensor surface at a consistent interval after cleaning, has been empirically demonstrated to significantly reduce both short and long term drift.

To combat the quenching of sensitivity under constant exposure, prior art systems include a “condition” cycle in control algorithms where the reading circuit is disabled and the heater (e.g., heater 144) is set to a temperature balanced below the MOx surface coating (e.g., surface coating 140) material damage onset temperature and the surface coating reset temperature, which may be hundreds of degrees Celsius. Prior art systems execute a condition cycle typically once per day. The result is that contaminants on the surface coating/sensor face are reset by, for example, evaporation or de-absorption/de-adsorption. Contaminants may include water collecting on the surface coating/sensor face, as water interferes with the resistivity of the MOx sensor. Contaminants may include chemicals, volatile organic compounds, and a myriad ambient pollutants in the atmosphere being measured. This conditioning cycle is typically recommended to combat long term drift, defined as drift in the MOx sensor sensitivity over a period of days.

Short term drift, which is drift observed on the order of minutes to hours, may be caused by any of the interactions between atmospheric species and the MOx sensor that have kinetics of accumulation or depletion in time scale of minutes to hours.

An objective of this system is to complement the long term drift conditioning with a method to reduce short term drift to quantitatively monitor levels of an antimicrobial gas in an environment where unacceptably high concentrations of the antimicrobial gas may jeopardize the safety of living beings present in the volume under treatment. The methods described both above and below are designed to both independently or simultaneously minimize MOx/SMOx sensitivity quenching and short to long term drift, from any of a variety of causes.

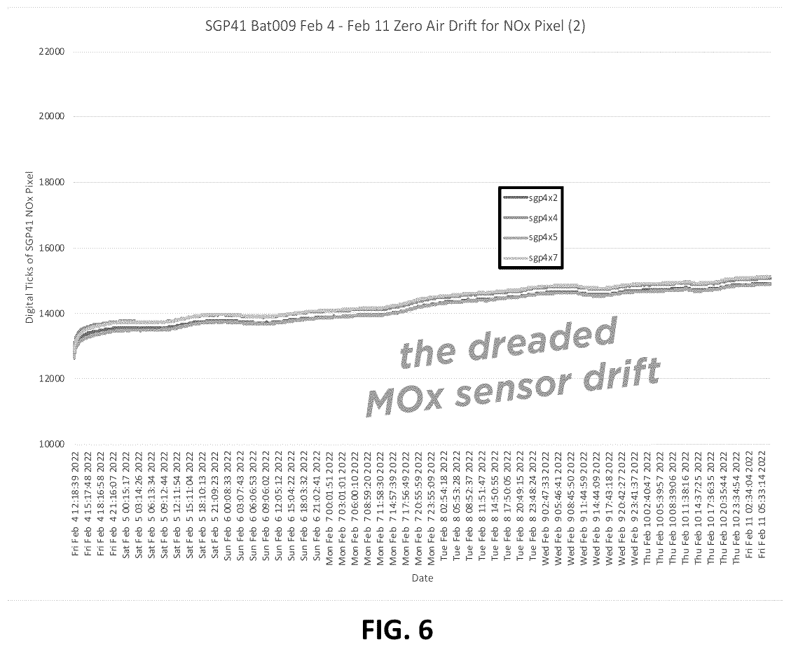

FIG. 6 illustrates a graph showing MOx sensor drift from sensor surface changes. The graph plots data over a seven day period showing long term MOx sensor drift (in four separate sensors) in a temperature and humidity controlled room, and the typical pattern of short term MOx sensor drift that limits the quantitative utility of MOx sensors (e.g., MOx sensor 102).

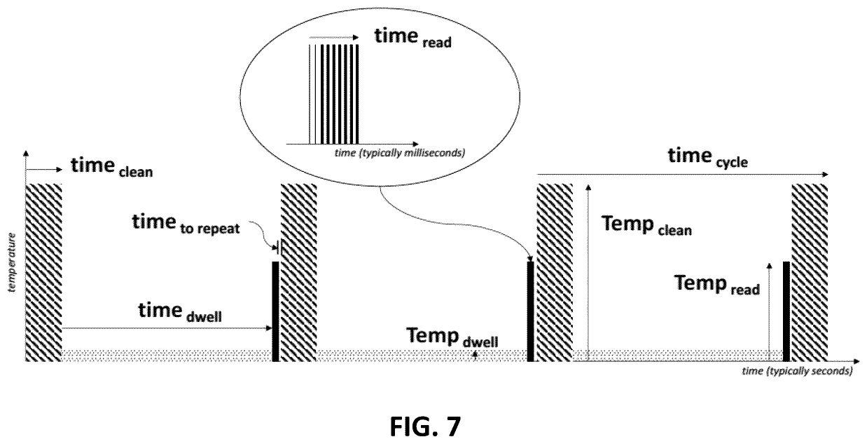

FIG. 7 illustrates a schematic of MOx sensor (e.g., MOx sensor 102) heater (e.g., heater 144) settings for clean-dwell-read (CDR) cycling of the sensor face (e.g., surface coating 140). To achieve objectives including minimizing sensitivity quenching on any time scale above seconds, and a unique method of using the re-population and MOx interaction kinetics of adsorbed/absorbed atmospheric components onto surface coating 140, the methods employed utilize readings gathered by the MOx sensor to coordinate the components described above via system control (e.g., microprocessor/microcontroller) to: (1) cause the heater to increase the sensor face temperature to a high temperature for a set period of time to prepare as clean a face as chemically possible (the clean portion of the cycle); (2) next, cause the heater to move to temperatures between the clean temperature and ambient in fixed or timed heat/cool patterns that are maintained for a (3) set holding period of time (the dwell portion of the cycle); then (4) wait the consistent interval of time dwell after clean; and (5) log the reading of the sensor in system control (the read portion of the cycle). The CDR cycle is constantly and continuously repeated throughout the period of time that the MOx sensor is deployed in an area to measure the concentration of a target gas (e.g., days, weeks, months, or years).

In the clean portion of the cycle, the heater is set to a temperature and duration sufficient to reset the MOx sensor face to a consistent starting point, but below levels that would change the surface coating's sintered material characteristics. This can be a static setting, or a time-based pattern of temperature setpoints, that provide a consistent baseline signal over time spans of minutes to years. For example, the cleaning time may be about 10 seconds in length. Also for example, the cleaning temperature may be about 500 degrees Celsius.

In the dwell portion of the cycle, a consistent specified length of time is allowed to pass. The dwell time and temperature may be varied in combinations and patterns. These combinations and patterns may be determined based upon the MOx sensor's return to baseline before the clean cycle was initiated. Some multiple or fraction of that baseline return time may be selected and used in the CDR cycle. The dwell temperature may be below the cleaning temperature, and may be at or below the reading temperature. The dwell temperature may be greater than the reading temperature. The dwell temperature may be ambient temperature, 200 degrees Celsius, 300 degrees Celsius, 400 degrees Celsius, and the like, as long as the dwell temperature is less than the read temperature. The dwell portion of the CDR cycle may be between about 2 seconds and about 600 seconds in length, measured from the end of the clean portion of the CDR cycle.

In the read portion of the cycle, the MOx sensor's reading of the change in electrical characteristics of the MOx surface due to exposure of the target gas is logged. MOx sensors need to be heated to the read temperature in order to work properly. The read temperature, or pattern of read temperatures, can be varied depending upon the analyte target. For example, the read cycle itself may be comprised of an average of millisecond to seconds reading times averaged or algorithmically discarded over seconds to hours of time. The read time may, for example, be between 2 seconds and 1200 seconds into the CDR cycle. Also for example, the read temperature may be about 150 degrees Celsius to 400 degrees Celsius. The heater may be capable of cycling the temperature of the sensor face between ambient temperature and the read temperature very quickly (e.g., in a matter of milliseconds), such that the temperature of the sensor face may quickly cycle back and forth between ambient and read temperatures as discussed below.

The read time may be selected based upon evidence that the MOx sensor has recovered almost entirely to baseline (more than 90%) to the mean by this time in the CDR cycle. Other read times may be selected based upon the sensor's recovery to baseline.

A repeat wait time may be added to the cycle after the read portion and may be set based upon the needs of frequency of sensing to either maximize the lifetime of the MOx sensor, preserve energy, and/or maintain the frequency of expected use case application gas concentration changes.

The total CDR cycle time may vary based upon a variety of parameters. For example, the CDR cycle time may be about four minutes in length (three cycles are illustrated in FIG. 7, for a total of about 12 minutes). As noted above, the CDR cycle may be repeated continuously (with or without a repeat wait time) throughout the life of the MOx sensor.

The CDR cycle may be used with an MOx sensor to combat sensor drift. In addition, the information gained from the sensor cycling through the CDR cycle may be useful in altering the operation of the system containing the MOx sensor. For example, the information may be used to reset the sensor baseline. In a closed loop gas delivery system, the CDR cycle could be used only during a programmatic cycle where the system delivering the gas has been maintain in a mode without gas delivery for a sufficient time to ensure concentration levels of the delivered analyte(s) have descended below the limits of detection. The CDR cycle may then be used to determine a new baseline of the system. In one aspect, the information gained in the CDR cycle may be used to change the modes of operation of the closed loop system, including diagnosing faults to raise a notification to an operator, confirmation that the system is within normal operating limits, reporting that something in the sensed environment is causing an error in the senor, and/or resetting of the baseline that ensures compensation for drift does not raise new safety concerns for the system.

A series of MOx sensors kept in different modes of operation than the CDR cycles, and the CDR cycles may be intermittently initiated to determine what is happening with the sensor system and/or the sensed environment.

An MOx/SMOx sensor may exhibit slow enough kinetics of recovery that are useful for computational gathering and fast enough to enable complex signal generation from the MOx/SMOx within an interval that is useful for monitoring air quality (e.g., within about five minutes).

As opposed to prior art embodiments that use electronic stimuli to generate multiple electronic signals from the MOx layer, the CDR cycle causes the physical action of heating via the system controller and MOx sensor subsystems. Further differentiation of the CDR cycle from the prior art are that what is measured are changes the MOx sensor's chemoresistive properties over time after the application of a thermal stimuli that consistently cleans the MOx sensor face of adsorbed/absorbed molecules.

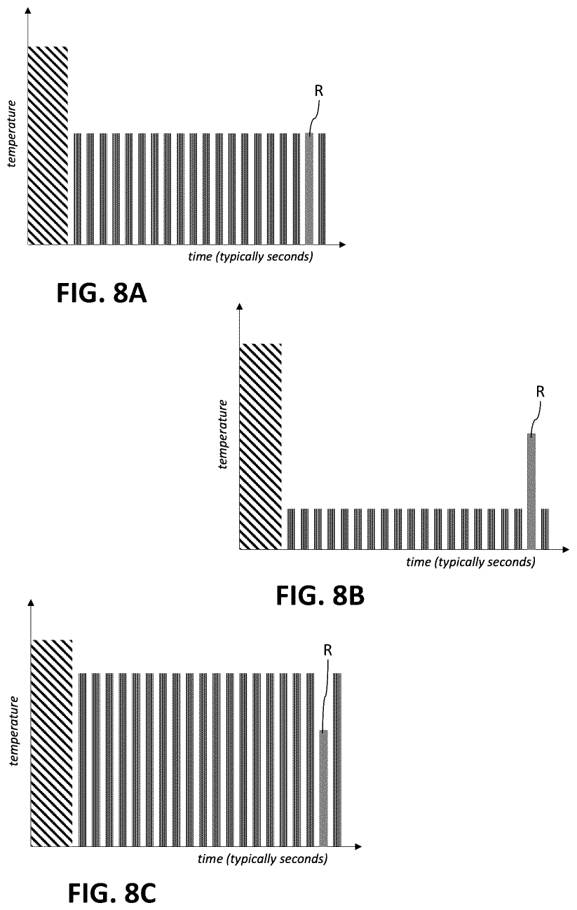

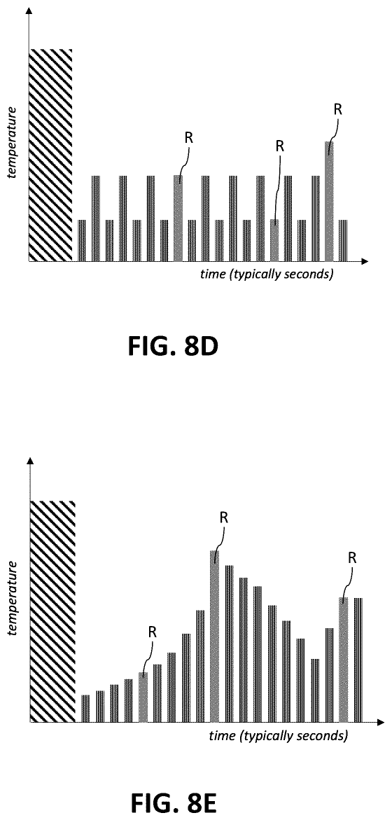

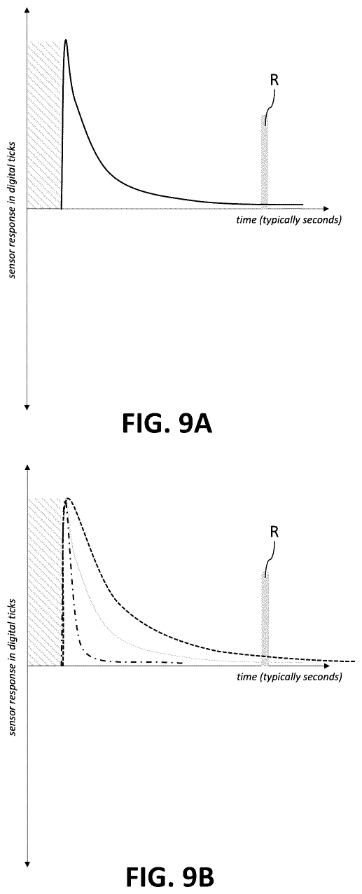

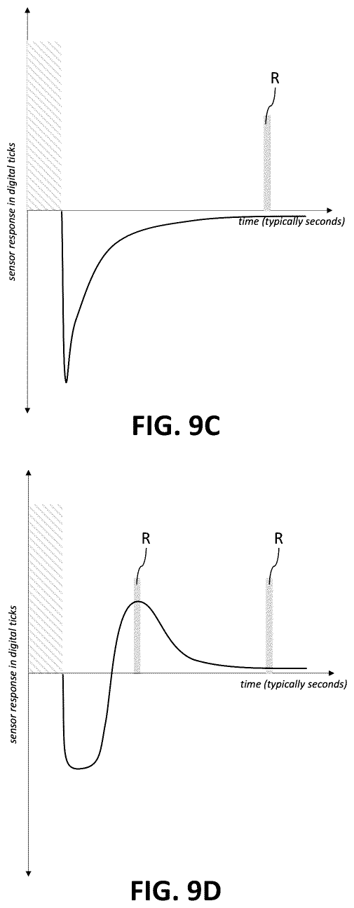

FIGS. 8A-8E illustrate example variations of clean-dwell-read cycling of the sensor face (e.g., surface coating 140). CDR cycle observations may assist in identifying the boundaries of thermal cycling in the MOx/SMOx sensor to measure sensor recovery interference reduction or acceleration rates as a means of getting computationally amenable software driven signals. Some possible thermal cycling signals of potential use may be achieved using the thermal stimulus patterns illustrated in FIGS. 8A-8E. These waveforms may result in sensor recovery illustrated in FIGS. 9A-9D, or similar. This analysis may focus on slope of decay following application of temperatures from about 300 degrees Celsius to about 500 degrees Celsius, time to recovery, sign of post-thermal cycling excursion (negative or positive) and whether it is different for reactive species that suppress or increase the MOx pixel reading, and the like.

The various thermal stimulus patterns illustrated in FIGS. 8A-8E represent possible programmable CDR cycles to be applied to an MOx sensor. The initial wide bar represents the clean portion of the cycle, where the temperature is increased (e.g., 500 degrees Celsius) and held for a period of time (e.g., about 10 seconds) as noted above. The following vertical bars represent the dwell portion of the cycle wherein the temperature is increased to a temperature less than the clean temperature (e.g., 200-400 degrees Celsius) for a short time (e.g., about two seconds), allowed to return to ambient for a short time (e.g., about one to two seconds), increased again, and so on for a period of about 218 seconds. Finally, the vertical bar denoted as R represents the read portion of the cycle and is the point at which a reading is taken and memorialized in the data. The read cycle point is selected to ensure that the sensor face has stabilized before that reading is memorialized in the data (e.g., at about 228 seconds from cycle initiation). A repeat wait time may follow the read portion of the cycle.

Specifically, FIG. 8A illustrates a CDR cycle in which a clean portion of the cycle may include a temperature of about 500 degrees Celsius held for about 10 seconds, followed by a dwell portion of the cycle in which the temperature is increased to about 300 degrees Celsius and decreased to ambient temperature repeatedly over the course of about 218 seconds. At about 228 seconds, the read portion R of the cycle includes increasing the temperature to about 300 degrees Celsius for about two seconds, and memorializing the reading of the MOx sensor at that point. A repeat wait time of a few seconds may follow the read portion of the cycle. FIG. 9A illustrates a possible stimulus response from the MOx sensor. As illustrated, the sensor has recovered to a long term baseline following the clean and dwell portions of the cycle before the read portion of the cycle is executed.

FIG. 8B illustrates a CDR cycle with a similar clean portion of the cycle as discussed with reference to FIG. 8A, but with a dwell portion in which the temperature is fluctuated between ambient and about 100 degrees Celsius over the dwell time period. The read portion R of the cycle includes raising the temperature to about 300 degrees Celsius as discussed with reference to FIG. 8A, followed by a repeat wait time of a few seconds before initiating the clean portion of the cycle once again. FIG. 9B illustrates a possible stimulus response from the MOx sensor. As illustrated, the sensor has recovered to a long term baseline following the clean and dwell portions of the cycle before the read portion of the cycle is executed. The upper and lower dashed lines may represent different chemical species of gases present. Any difference between the slopes of the lines may help teach the system how to more readily identify the target gas and its concentration.

FIG. 8C illustrates a CDR cycle with a similar clean portion of the cycle as discussed with reference to FIG. 8A, but with a dwell portion in which the temperature is fluctuated between ambient and about 400 degrees Celsius over the dwell time period. The read portion R of the cycle includes raising the temperature to about 300 degrees Celsius as discussed with reference to FIG. 8A, followed by a repeat wait time of a few seconds before initiating the clean portion of the cycle once again. FIG. 9C illustrates a possible stimulus response from the MOx sensor. As illustrated, the sensor has recovered to a long term baseline following the clean and dwell portions of the cycle before the read portion of the cycle is executed. The sign of the excursion may change based upon a reactive species that is oxidizing or reducing. Some gases tend to be oxidizing while other gases tend to be reducing. As a result, a sign change may be evident in the presence of specific gases.

FIG. 8D illustrates a CDR cycle with a similar clean portion of the cycle as discussed with reference to FIG. 8A, but with a dwell portion in which the temperature is fluctuated between ambient and about 100-200 degrees Celsius over the dwell time period. Rather than executing a single read portion R of the cycle, three independent read portions R may be executed and at varying read temperatures (e.g., 100, 200, and 300 degrees Celsius), with additional dwell portions contained between the read portions R. The final read portion R may be followed by a repeat wait time of a few seconds before initiating the clean portion of the cycle once again. FIG. 9D illustrates a possible stimulus response from the MOx sensor. The recovery slope observed by the various read portions R may help identify a particular observed gas as the target gas, thus confirming that the MOx sensor is indeed observing the appropriate gas.

FIG. 8E illustrates a CDR cycle with a similar clean portion of the cycle as discussed with reference to FIG. 8A, but with a dwell portion in which the temperature is fluctuated between ambient and about 75-350 degrees Celsius over the dwell time period. Rather than executing a single read portion R of the cycle, three independent read portions R may be executed and at varying read temperatures (e.g., 100, 300, and 400 degrees Celsius), with additional dwell portions contained between the read portions R. The final read portion R may be followed by a repeat wait time of a few seconds before initiating the clean portion of the cycle once again. Similar to the CDR cycle noted in FIG. 8D, the recovery slope observed by the various read portions R may help identify a particular observed gas as the target gas, thus confirming that the MOx sensor is indeed observing the appropriate gas.

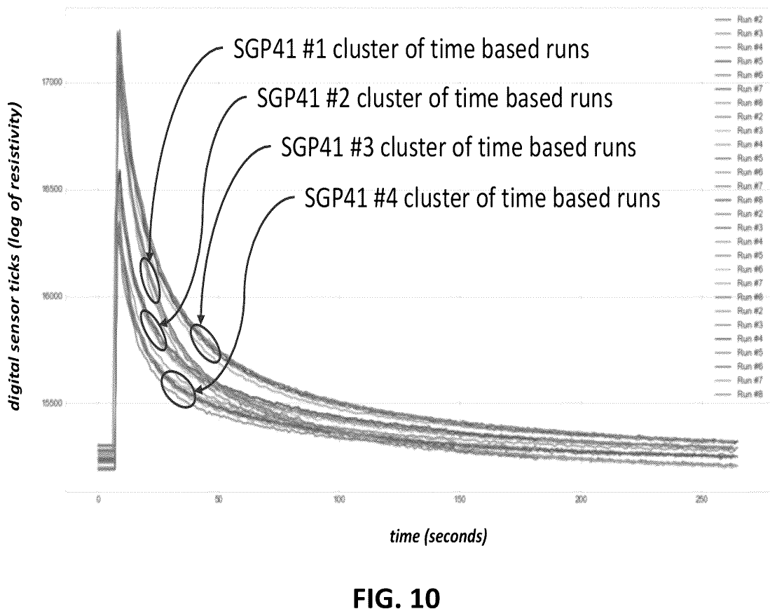

FIG. 10 illustrates a graph plotting the time-based recovery of the surface of an MOx sensor to long term baseline after a condition cycle has been completed until 275 seconds has passed. The curves shown are generated by collecting digital sensor ticks reported across a repeated series of about two second read cycles (at about 300 degrees Celsius) for the entire duration of the dwell portion of the CDR cycle. This data illustrates the time required to reset the MOx sensor to a long term baseline in a typical room atmosphere. The data was collected using one board, with four sensors, in an atmosphere without any introduced reactive gas components. In about the first 10 seconds plotted in the graph the MOx sensor face transitions from ambient temperature to the clean portion temperature (e.g., about 500 degrees Celsius), during which adsorbed/absorbed species are removed from the sensor face. At this elevated temperature, water, oxygen, and other present gas species are collectively affecting the resistivity measurement across the sensor. After about 175 seconds has elapsed, the MOx sensor shows nearly full recovery (greater than 90% recovery) of the long term baseline.

While prior art systems may measure electronic signals while applying electronic a/c waveform stimulations, the CDR cycle concept is measuring a physical (in this case thermal) stimulus and measuring chemoresistive changes due to repopulation of environmental chemical species on the MOx sensor face. This decay may be unperturbed decay or intentionally perturbed decay.

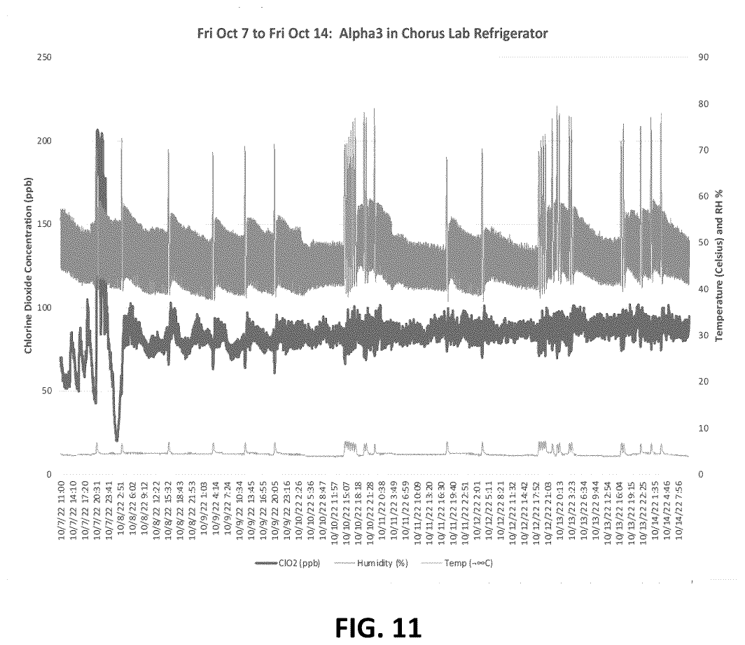

FIG. 11 illustrates a graph showing MOx sensor stability over seven days of a stability experiment. Application of the CDR cycle illustrates that the MOx sensor, while in the presence of ClO₂, exhibits no short term or long term sensor drift. The data in the graph was collected over a 10 day period. Coupled with a ClO₂ generator, the sensor was able to control a 4.3 cubic meter commercial refrigerator environment, averaging 4 degrees Celsius, at an average of 85 ppb of ClO₂ for a 10 day food preservation experiment. Both the stability in “digital ticks” and the corresponding conversion of ticks to ppb by an internal calibration table confirms that sensor drift did not occur. The middle curve is the measurement of ClO₂ in ppb. The upper curve is the relative humidity of the refrigerator. The lower curve is the temperature of the refrigerator.

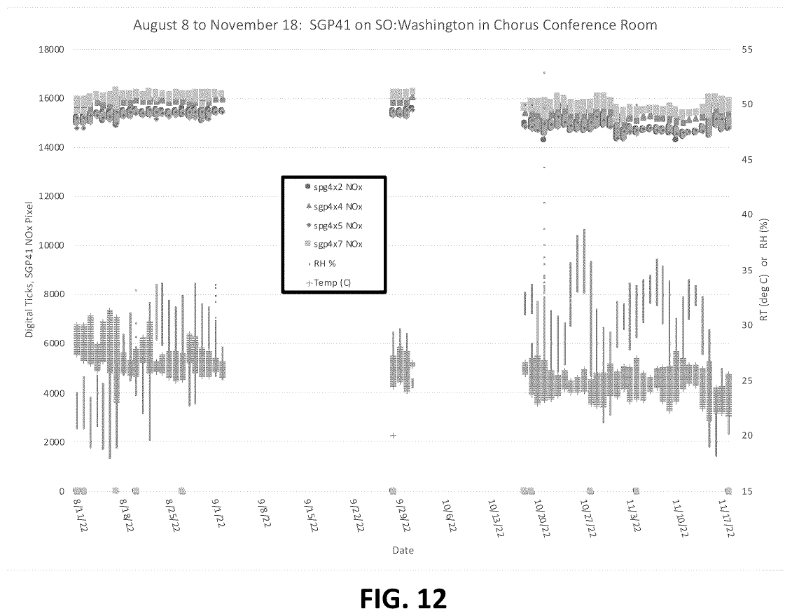

FIG. 12 illustrates a graph showing MOx sensor stability as a result of conditioning cycles. After implementing the CDR cycle in the MOx sensor, long term drift is eliminated allowing the expected sensor changes to environmental interferents to be accurately measured. The digital ticks collected by the MOx sensors are illustrated in the upper curves of the graph. The lower curves of the graph illustrate the temperature and humidity in the sensed volume.

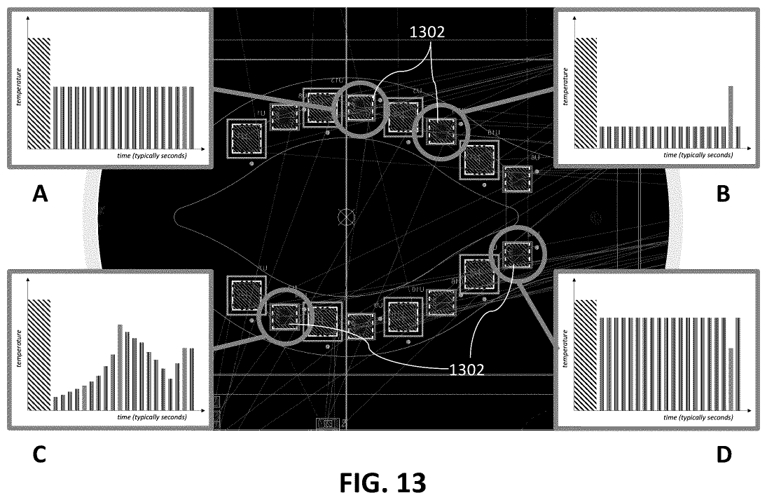

FIG. 13 illustrates an array of MOx sensors 1302, each using a different variation of CDR cycling of the sensor face. The system can compare the readings from the different sensors 1302 to identify multiple unknown gases. In one aspect, the system can use machine learning to compare the readings from the different sensors 1302 to make these determinations. The use of different sensors 1302 with different CDR cycles may provide differing ratios of sensitivities with different gases, and when applied to machine learning algorithms, may result in different outputs specific to each gas. In this manner, the system can either weed out non-target gases, collect concentration data on multiple target gases, or both.

The use of different sensors 1302 with different CDR cycles may provide differing ratios of sensitivities with different gases, and may provide increased accuracy when identifying the concentration of a single target gas. The different sensors 1302 may each give a unique perspective of the concentration of the target gas, which when combined with other sensors, may permit the focusing of the system on the target gas and the weeding out of the non-target gases. The system may allow slight discrimination to full discrimination and/or quantification to focus upon the concentration of the target gas.

Additionally, room temperature and room humidity sensors may be used in conjunction with any of the systems described herein to provide a more accurate measurement of the concentration of the target gas within the room. Additionally, a separate CO₂ sensor may be used in conjunction with any of the systems described herein (with or without room temperature and room humidity sensors) so that a known concentration of CO₂ can be factored into the data related to the target gas concentrations. That is, data collected on the target gas concentrations can be adjusted based upon known effects of room temperature, room humidity, and/or CO₂ concentration upon that data.

As an example of a specific use case, the systems described herein, using MOx/SMOx sensors, may be used to monitor the concentration of an antimicrobial gas, such as ClO₂, in a volume under treatment. Accurate measurement of the concentration of the antimicrobial gas may be critical, particularly in cases where living beings are present in the volume under treatment. In such a scenario, an accepted safe concentration of antimicrobial gas must not be exceeded in order to protect the health and safety of those living beings. The system and sensors described herein may monitor the concentration of antimicrobial gas and in turn cause the antimicrobial gas generator to generator more or less of the antimicrobial gas, and/or issue an alarm where the accepted safe concentration of the antimicrobial gas is exceeded.

In one aspect, a method for increasing the consistency of MOx sensor readings over an in-service interval spanning minutes to years is provided, the method comprising: applying a thermal stimulus to a MOx sensor face to reset the sensor face to a consistent condition that is devoid of environmental chemical species that accumulated on the sensor face in the past; a dwell period following the application of the thermal stimulus where the MOx sensor is at least one of: (a) held at a temperature between an ambient temperature and a thermal stimulus temperature, or (b) exposed to temporal patterns of heated and unheated intervals; triggering a reading of electrochemical changes to the MOx sensor caused by chemical species in the environment being sensed by the MOx sensor, at a consistent time interval after the application of the thermal stimulus; and wherein the application of the thermal stimulus, the following dwell period, and the reading of electrochemical changes to the MOx sensor is a single thermal stimulus-to-read cycle. The MOx sensor may be in a continuous mode of operation where once a thermal stimulus-to-read cycle is completed, a subsequent thermal stimulus is initiated to begin a subsequent thermal stimulus-to-read cycle. The MOx sensor thermal stimulus-to-read cycle may be followed by a dwell time interval where the MOx sensor is in a dwell mode of operation, and upon the cessation of that dwell time interval a subsequent thermal stimulus-to-read cycle is initiated. The method may include a system controller synchronizing or coordinating readings of an electronic signal caused by interactions of the MOx sensor with the chemical species in the environment at a consistent time interval following the application of the thermal stimulus. The system controller may synchronize or coordinate readings of a change in electrical resistance caused by interactions of the MOx sensor with the chemical species in the environment at a consistent time interval following the application of the thermal stimulus. The method may include a computational system that receives the readings of the electronic signal for computation, conversion, qualitative or quantitative expressions representative of the readings. The method may include a memory system wherein the electronic signal and/or change in electrical potential is stored in the memory system for temporal storage.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See Bryan A. Garner, A Dictionary of Modern Legal Usage 624 (2d. Ed. 1995). Also, to the extent that the terms “in” or “into” are used in the specification or the claims, it is intended to additionally mean “on” or “onto.” To the extent that the term “substantially” is used in the specification or the claims, it is intended to take into consideration the degree of precision available in manufacturing. To the extent that the term “selectively” is used in the specification or the claims, it is intended to refer to a condition of a component wherein a user of the apparatus may activate or deactivate the feature or function of the component as is necessary or desired in use of the apparatus. To the extent that the term “operatively connected” is used in the specification or the claims, it is intended to mean that the identified components are connected in a way to perform a designated function. As used in the specification and the claims, the singular forms “a,” “an,” and “the” include the plural. Finally, where the term “about” is used in conjunction with a number, it is intended to include ±10% of the number. In other words, “about 10” may mean from 9 to 11.

As stated above, while the present application has been illustrated by the description of aspects thereof, and while the aspects have been described in considerable detail, it is not the intention of the applicants to restrict or in any way limit the scope of the appended claims to such detail. Additional advantages and modifications will readily appear to those skilled in the art, having the benefit of the present application. Therefore, the application, in its broader aspects, is not limited to the specific details, illustrative examples shown, or any apparatus referred to. Departures may be made from such details, examples, and apparatuses without departing from the spirit or scope of the general inventive concept.

What is claimed is:

1. A method for increasing the consistency of MOx sensor readings over an in-service interval spanning minutes to years, comprising:
applying a thermal stimulus to a MOx sensor face to reset the sensor face to a consistent condition that includes removal of accumulated environmental chemical species from the sensor face;
a dwell period following the application of the thermal stimulus where the MOx sensor is at least one of: (a) held at a consistent temperature between an ambient temperature and a thermal stimulus temperature, or (b) exposed to temporal patterns of heated and unheated intervals;
triggering a reading of electrochemical changes to the MOx sensor caused by chemical species in the environment being sensed by the MOx sensor, at a consistent time interval after the application of the thermal stimulus; and
wherein the application of the thermal stimulus, the following dwell period, and the reading of electrochemical changes to the MOx sensor is a single thermal stimulus-to-read cycle.

2. The method of claim 1, wherein the MOx sensor is in a continuous mode of operation where once a thermal stimulus-to-read cycle is completed, a subsequent thermal stimulus is initiated to begin a subsequent thermal stimulus-to-read cycle.

3. The method of claim 1, wherein the MOx sensor thermal stimulus-to-read cycle is followed by a dwell time interval where the MOx sensor is in a dwell mode of operation, and upon the cessation of that dwell time interval a subsequent thermal stimulus-to-read cycle is initiated.

4. The method of claim 1, further comprising a system controller synchronizing or coordinating readings of an electronic signal caused by interactions of the MOx sensor with the chemical species in the environment at a consistent time interval following the application of the thermal stimulus.

5. The method of claim 4, wherein the system controller synchronizes or coordinates readings of a change in electrical resistance caused by interactions of the MOx sensor with the chemical species in the environment at a consistent time interval following the application of the thermal stimulus.

6. The method of claim 4, further comprising a computational system that receives the readings of the electronic signal for computation, and stores the electronic signal in a memory system for temporal storage.

7. The method of claim 5, further comprising a computational system that receives the readings of the change in electrical resistance for computation, and stores the change in electrical resistance in a memory system for temporal storage.

8. The method of claim 4, further comprising a computational system that receives the readings of the electronic signal for conversion, and stores the electronic signal in a memory system for temporal storage.

9. The method of claim 5, further comprising a computational system that receives the readings of the change in electrical resistance for conversion, and stores the change in electrical resistance for temporal storage.

10. The method of claim 4, further comprising a computational system that receives the readings of the electronic signal for qualitative expressions representative of the readings, and stores the electronic signal in a memory system for temporal storage.

11. The method of claim 5, further comprising a computational system that receives the readings of the change in electrical resistance for qualitative expressions representative of the readings, and stores the change in electrical resistance for temporal storage.

12. The method of claim 4, further comprising a computational system that receives the readings of the electronic signal for quantitative expressions representative of the readings, and stores the electronic signal in a memory system for temporal storage.

13. The method of claim 5, further comprising a computational system that receives the readings of the change in electrical resistance for quantitative expressions representative of the readings, and stores the change in electrical resistance for temporal storage.

14. A system for controlling and coordinating a stimulus-to-read cycle of an MOx sensor, comprising:
a metal oxide sensor having:
a substrate supporting a sintered powder MOx surface coating,
one or more electrode electrically connected to the surface coating,
a heater, and
a power source;
a heater controller; and
a system controller;
wherein the system controller is operatively connected to the heater controller and configured to cause the heater to input heat to the surface coating in a continuous cycle of clean, dwell, and read actions,
wherein the clean action includes heating the surface coating to a temperature sufficient to remove environmental chemical species accumulated on the surface coating but below a level that would change a sintered material characteristic of the surface coating,
wherein the dwell action includes periodically heating the surface coating to a temperature less than the clean action temperature, and
wherein the read action includes heating the surface temperature to a temperature that is less than the clean action temperature.

15. The system of claim 14, wherein the target gas is an antimicrobial gas.

16. The system of claim 14, wherein a plurality of read actions are executed during the continuous cycle, and wherein the plurality of read actions occur between a plurality of dwell actions.

17. A method for identifying a target gas concentration using an MOx sensor, comprising:
providing a metal oxide sensor having:
a substrate supporting a sintered powder MOx surface coating,
one or more electrode electrically connected to the surface coating,
a heater, and
a power source;
providing a heater controller; and
providing a system controller;
wherein the system controller is operatively connected to the heater controller and causes the heater to input heat to the surface coating in a continuous cycle of clean, dwell, and read actions,
wherein the clean action includes heating the surface coating to a temperature sufficient to remove environmental chemical species accumulated on the surface coating but below a level that would change a sintered material characteristic of the surface coating,
wherein the dwell action includes periodically heating the surface coating to a temperature that is less than the clean action temperature, and
wherein the read action includes heating the surface temperature to a temperature greater than the dwell action temperature and less than the clean action temperature.

18. The method of claim 17, wherein the target gas is an antimicrobial gas.

19. The method of claim 17, wherein a plurality of read actions are executed during the continuous cycle, and wherein the plurality of read actions occur between a plurality of dwell actions.

20. The method of claim 17, wherein the target gas is an oxidizing gas.

21. The method of claim 17, wherein the target gas is a reducing gas.
